SEQUENCE LISTING (1 sequence)

SEQ ID NO: 1 - DNA, 13 bp, Artificial Sequence
  Description of Artificial Sequence Synthetic oligonucleotide
aaaatttcccggg

(19) United States
(12) Patent Application Publication
(10) Pub. No.: US 2019/0323050 A1
(43) Pub. Date: Oct. 24, 2019

(54) Modulation of Enzymatic Polynucleotide Synthesis Using Chelated Divalent Cations

(71) Applicant: President and Fellows of Harvard College, Cambridge, MA (US)

(72) Inventors: Kettner John Frederick Griswold, Jr., Brookline, MA (US); Howon Lee, Allston, MA (US); George M. Church, Brookline, MA (US)

(73) Assignee: President and Fellows of Harvard College, Cambridge, MA (US)

(21) Appl. No.: 16/472,268

(22) PCT Filed: Dec. 21, 2017
(86) PCT No.: PCT/US17/67931

§ 371 Date: Jun. 21, 2019

Related U.S. Application Data
(60) Provisional application No. 62/437,349, filed on Dec. 21, 2016.

(51) Int. Cl. C12P 19/34; C12N 9/12

(52) U.S. Cl. CPC C12P 19/34; C12N 9/1264

(57) ABSTRACT

Methods and apparatus of modulating polynucleotide synthesis are provided. The methods include delivering reagents comprising enzymes, nucleotides and ions to oligonucleotide primers wherein the reagents catalyze incorporation of the nucleotides to 3′ ends of the oligonucleotide primers, and modulating incorporation of the nucleotides to the 3′ ends of the oligonucleotide primers. Polynucleotide synthesis is modulated by modulating presence or absence of catalytic cation cofactors to provide sequence defined synthesis of polynucleotides. In certain embodiments, the polynucleotides encode information.

### RELATED APPLICATION DATA

This application claims priority to U.S. Provisional Application 62/437,349 filed on Dec. 21, 2016, which is hereby incorporated herein by reference in its entirety for all purposes.

### STATEMENT OF GOVERNMENT INTERESTS

This invention was made with government support under grant numbers RM1HG008525 and R01MH103910 awarded by National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on Jan. 24, 2018, is named 010498_01022 WO_SL.txt and is 558 bytes in size.

### FIELD

The present invention relates in general to methods of making oligonucleotides and polynucleotides using enzymatic synthesis.

### BACKGROUND

Methods of polynucleotide synthesis using template-independent polymerases are known. However, a need exists for modulating the enzymatic synthesis of polynucleotides.

### SUMMARY

According to one aspect, the present disclosure provides a method of modulating enzymatic polynucleotide synthesis using a template independent polymerase, a selected nucleotide, and one or more cations, wherein the one or more cations can be in an active or inactive state. When the one or more cations are in an active state, the template independent polymerase catalyzes addition of a selected nucleotide to a target substrate for the template independent polymerase. According to one aspect, the one or more cations are active when in a free cation state and are in a reaction region capable of participating in the enzymatic addition reaction. The one or more cations can be in an inactive state when bound by a chelator compound and therefore the cation is not in a free state. According to one aspect, the bound cation is in an inactive state, but can be activated by being released from the chelator. The active cation may then participate in an enzymatic nucleotide addition reaction. Free cation may then be bound by a chelator present in the reaction medium in a molar excess thereby rendering it inactive and terminating the enzymatic nucleotide addition reaction.

The one or more cations may be in a precursor state, such as being in a zero valent state or a reduced state. The precursor may be oxidized to produce free cation which may then participate in an enzymatic nucleotide addition reaction. Free cation may then be reduced to a zero valent state or reduced state thereby rendering it inactive and terminating the enzymatic nucleotide addition reaction.

The one or more cations may be introduced into the reaction region to be available for participating in the enzymatic nucleotide addition reaction or they may be transported away from the reaction region thereby terminating the enzymatic nucleotide addition reaction. Methods of creating or transporting cations are known to those of skill in the art.

Further features and advantages of certain embodiments of the present invention will become more fully apparent in the following description of embodiments and drawings thereof, and from the claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The foregoing and other features and advantages of the present embodiments will be more fully understood from the following detailed description of illustrative embodiments taken in conjunction with the accompanying drawings in which:

FIG. 1 depicts a schematic of using light to control release of a cation bound to a photoactive chelator. When the cation is in the unbound or active state, an enzymatic primer extension reaction takes place whereby TdT enzymatically adds a nucleotide to a target substrate for the enzyme. When light is not applied, excess chelator in the reaction medium binds to unbound cations thereby rendering them in the inactive state.

FIG. 2 depicts photo uncaging or releasing of an exemplary caged or bound cation using light where DMNP-EDTA is photocleaved.

FIG. 3 depicts a schematic of an exemplary process for continuous flow DNA synthesis.

FIG. 4 depicts a schematic of an exemplary apparatus for enzymatic DNA synthesis controlled by light using light cleavable chelator compounds.

FIG. 5 depict gel studies showing selective TdT enzymatic elongation resulting from UV uncaging of Co⁺² and Mg⁺².

FIG. 6A shows components and conditions for the experiments of Example II.

FIG. 6B depicts gel studies showing selective TdT enzymatic elongation resulting from UV uncaging of Co⁺².

FIG. 7A depicts a general experimental patterned UV illumination experiment and reaction components and conditions.

FIG. 7B shows a square fluorescence pattern resulting from selective activation of TdT enzyme on the surface of glass slide.

FIG. 8A depicts in schematic a splint ligation method.

FIG. 8B depicts results of experiments where splint ligation was used to ligate a labeled probed to a ssDNA having a 4 nucleotide or greater homopolymer end sequence using a splint oligonucleotide sequence having a 4 nucleotide homopolymer complementary sequence.

### DETAILED DESCRIPTION

The present disclosure is directed to a method and apparatus for enzymatically synthesizing an oligonucleotide sequence or polynucleotide sequence, such as a single stranded oligonucleotide sequence or polynucleotide sequence, whether random or designed using ion modulation to activate or inactivate the enzymatic synthesis reaction. Other methods of modulating the enzymatic synthesis reaction are described herein and may be used alone or in combination with methods of modulating ions to control the enzymatic synthesis reaction. Polymerase extension of polynucleotides is understood in the art to require one or more cations, such as at least two metal divalent ions, in the mechanism for catalysis of phosophodiester bond formation. According to one aspect, an enzyme, a nucleotide, and an inactive cation or cations (and related reagents or conditions) are placed at a reaction region or reaction site under appropriate reaction conditions. The cation or cations are activated and the nucleotide is enzymatically and covalently bound to an existing 3′ nucleotide, such as that on an initiator sequence or nucleic acid sequence as part of a substrate for the enzyme. The substrate for the enzyme may be attached to a support. The cation or cations may be inactivated to terminate the enzymatic nucleotide addition reaction.

A plurality of oligonucleotide sequences may be created in a multiplexed method of delivering reagents to various reaction regions and activating the cations to add one or more nucleotides followed by deactivating the cations. The oligonucleotide sequences may be synthesized using polymerases, such as error-prone polymerases under conditions where the reagents are localized at a location on a substrate for a period of time and under such conditions where the cation is activated and deactivated to maximize probability of adding a single nucleotide or desired number of nucleotides. A suitable wash may also be used at a desired time to remove one or more reagents from the reaction site or location. The reagents or wash may be added to a location or reaction site using any suitable fluidics system or other systems known to those of skill in the art.

According to one aspect, an enzyme, a nucleotide, and a cation or cations (and related reagents or conditions) are placed at a reaction region or reaction site under appropriate reaction conditions. The nucleotide is enzymatically and covalently bound to an existing 3′ nucleotide, such as that on an initiator sequence or nucleic acid sequence as part of a substrate for the enzyme. The substrate for the enzyme may be attached to a support. The cation or cations may be inactivated to terminate the enzymatic nucleotide addition reaction.

Cations may be in the inactive form when bound to a chelator compound. Cations may be in the active or free form after being released from the chelator compound or they may be provided to the reaction region in the active or free form. Alternatively, cations may be created from zero valent or reduced materials using oxidation methods known to those of skill in the art. cations may also be transported to a reaction region and away from a reaction region. Once the cation has participated in the enzymatic reaction as described herein, the free cation may be inactivated by being bound to a chelator compound which is either in molar excess in the reaction medium or provided to the reaction medium in an amount sufficient to bind or inactivate the cation so that the enzymatic nucleotide addition reaction is terminated. Exemplary chelator compounds may be readily identified based on the present disclosure as those which bind to a cation and then may release the cation in response to a stimulus such as light, heat, pH, a chemical, electrons, an electrical potential, etc., as is known to those of skill in the art. The cation may also be inactivate by being reduced using methods known to those of skill in the art. the cation may also be transported away from the reaction region.

According to one aspect, altering the state of catalytic ions can be used to modulate enzymatic single stranded polynucleotide synthesis. In certain embodiments, the catalytic ion cofactor can be reduced or oxidized photochemically or electrochemically to modulate extension by a template independent polymerase. In one embodiment, divalent ions may be reduced or reduced or zero valent materials may be oxidized by electrodes. In one embodiment of an electrochemical multiplex control device, divalent ions may be reduced or reduced or zero valent materials oxidized by electrodes independently controlled in an array. Electron delivery systems described herein can be used in the control of the oxidation state of atoms useful in the present methods.

In an alternative embodiment, catalytic ions can be rapidly transported away from an enzyme in a reaction region fixed in space by electrophoresis or electroosmosis. In one embodiment of a control device such as a multiplexed control device, electrode arrays are used such that AC or DC electric fields normal to the surface of the polynucleotide support drive ions away from the growing polynucleotide in select locations in an array. Electron delivery systems described herein can be used in the control of the reaction array.

In one embodiment, the catalytic ion (cation or divalent cation) is competitively inhibited from occupying the active site of the template independent DNA polymerase by ion chelating agents. For example, EDTA and ion chelating agents can competitively inhibit enzyme activity by reducing the concentration of available free ion. In one embodiment, electrochemical cleavage of ion chelating agents provide free cation concentration at one or more reaction regions, for example at desired locations on an array support, such as with a multiplex modulation and synthesis method. Electron delivery systems described herein can be used in the control of the reaction array. In a separate embodiment of the disclosure, a photolabile “caged” chelator is used. The term “caged” as used herein refers to a molecule containing a photolabile moiety that upon cleavage returns the active form of the caged molecule. The terms “chelator,” “chelating agent,” or “chelating compound” are used herein to describe compounds, such as polydentate ligands, capable of forming coordinate complexes with ions having a plurality of coordinate bonds. Chelation compounds are known in the art and may include, but are not limited to, any of the of the following molecules: aminophenol-N,N,O-triacetate (APTRA), (1,2-bis(o-aminophenoxy)ethane-N,N,N′,N′-tetraacetic acid) (BAPTA), bipyridine (bpy), 1,4,8,11-tetraazacyclotetradecane (cyclam), desferrioxamine (DFO), 1,4,7,10-tetraazacyclododecane-1,4,7,10-tetraacetic acid (DOTA), dipyridylamine (DPA), diethylenetriamine pentaacetic acid (DTPA), ethylene diamine tetraacetic acid (EDTA), ethylene glycol tetraacetic acid (EGTA), nitrilotriacetic acid (NTA), and N,N,N′,N′-tetrakis-(2-pyridylmethyl)-ethylenediamine (TPEN), or other molecules. Chelators are well known to those skilled in the art, and they are reviewed extensively in Kathryn L. Haas and Katherine J. Franz, Application of Metal Coordination Chemistry to Explore and Manipulate Cell Biology, Chem Rev. 2009 October; 109(10): 4921-4960, doi: 10.1021/cr900134a, hereby incorporated by reference in its entirety. According to one aspect, one or more chelator compounds are provided in molar excess in the reaction medium to bind or sequester ions, such as cations, available in a reaction medium thereby rendering them inactive and unable to participate in an enzymatic nucleotide addition reaction thereby inhibiting enzymatic activity.

Caging groups are known in the art and may include, orthonitrobenzyl derivatives, otho-nitroindole derivatives and others and are described in Graham C R Ellis-Davies, Caged compounds: photorelease technology for control of cellular chemistry and physiology, Nat Methods, 2007 August; 4(8): 619-628, doi: 10.1038/nmeth1072 and S R Adams and R Y Tsien, Controlling Cell Chemistry with Caged Compounds, Annual Review of Physiology, Vol. 55: 755-784, (1993), DOI: 10.1146/annurev.ph.55.030193.003543, hereby incorporated by reference in their entireties.

Photolabile derivatives of known high-affinity cation chelators (BAPTA, EDTA and EGTA) have been synthesized previously and are known to those of skill in the art. An exemplary caged cation reagent is 1-(4,5-dimethoxy-2-nitrophenyl) EDTA (DMNP-EDTA). Upon illumination at 365 nm, UV light induces bond cleavage in the ligand backbone, which reduces the denticity of the ligands. DMNP-EDTA's affinity for divalent cations decreases irreversibly by >600,000-fold upon cleavage. Thus, photolysis of DMNP-EDTA complexed with divalent cations results in production of free divalent cations. Caged cation chelators may be used as a mechanism for control of ionic cofactor dependent reactions. Caged cation cofactors may be useful in the present disclosure for the control of template independent DNA polymerase in the synthesis of polynucleotides. According to one aspect, one or more cations bound to a light activated or photo inducible or photo sensitive chelator compound or compounds are provided to a reaction region or are present at a reaction region and the reaction region is illuminated with light of a particular wavelength sufficient to cause the chelator compound or compounds to release the one or more cations. This disclosure provides photon delivery methods for releasing or uncaging of molecules or cations useful in the present disclosure.

Polymerases, including without limitation error-prone template-dependent polymerases, modified or otherwise, can be used to create nucleotide polymers having a random or known or desired sequence of nucleotides. Template-independent polymerases, whether modified or otherwise, can be used to create the nucleic acids de novo. Ordinary nucleotides are used, such as A, T/U, C or G. The disclosure provides for the use of chain terminating moieties on nucleotides. Such nucleotides with chain terminating moieties may be referred to as reversible terminators. The disclosure also provides for the use of nucleotides which lack chain terminating moieties. A template independent polymerase may be used to make the nucleic acid sequence. Such a template independent polymerase may be error-prone which may lead to the addition of more than one nucleotide resulting in a homopolymer. Sensors, such as light activated sensors, metabolic products or chemicals, that are activated by ligands can be used with such polymerases.

Oligonucleotide sequences or polynucleotide sequences are synthesized using an error prone polymerase, such as template independent error prone polymerase, and common or natural nucleic acids, which may be unmodified. Initiator sequences or primers that are substrates for the polymerase are attached to a support, such as a silicon dioxide substrate, at various locations whether known, such as in an addressable array, or random. Methods and moieties for attaching nucleotide sequences to a support are well known in the art. Such moieties may be cleavable such that the resultant oligonucleotide may be cleaved from the support surface, for example, by chemical reagents or light. Reagents including at least a selected nucleotide, a template independent polymerase and other reagents required for enzymatic activity of the polymerase are applied at one or more locations of the substrate where the initiator sequences are located and under conditions where the polymerase adds one or more than one or a plurality of the nucleotide to the enzyme substrate which may be an initiator sequence or an existing oligonucleotide or polynucleotide. The nucleotides (such as nucleotide triphosphates or “dNTPs”) may be applied or flow in periodic applications. Blocking groups or reversible terminators may be used with the dNTPs. Nucleotides with blocking groups or reversible terminators are known to those of skill in the art. According to an additional embodiment when reaction conditions permit, more than one dNTP may be added to form a homopolymer run when common or natural nucleotides are used with a template independent error prone polymerase. When blocking groups or reversible terminators are used, the blocking group or terminating group is removed which allows extension of the growing oligonucleotide by addition of the next nucleotide. According to methods described herein, the state of active cations is modulated to facilitate extension of the growing oligonucleotide by addition of the next nucleotide. Other modulation methods are described herein.

Polymerase activity may be modified using photo-chemical or electrochemical modulation of catalytic cations as a reaction condition so as to minimize addition of dNTP beyond a single dNTP. Cations may be modulated within a reaction region between an active and an inactive state to promote activity of or inhibit activity of the polymerase. A wash may be applied to the one or more locations or reaction regions to remove the reagents. The steps of applying the reagents and the wash are repeated until desired nucleic acids are created. The reagents may be added to one or more than one or a plurality of locations on the substrate in series or in parallel or the reagents may contact the entire surface of the support, such as by flowing the reagents across the surface of the support. Reaction conditions for adding a nucleotide using an enzyme are known to those of skill in the art and may be readily determined.

In addition, according to certain embodiments, polymerases can be modulated to be light sensitive for light based methods. According to this aspect, light is modulated to tune the polymerase to add a nucleotide or a number of nucleotides when the polymerase is illuminated with the light of certain wavelength. The light is shone or illuminated on individual locations or pixels of the substrate where the polymerase, the nucleotide and appropriate reagents and reaction conditions are present. In this manner, a nucleotide is added to an initiator sequence or an existing nucleotide as the polymerase is activated by the light. Suitable illumination systems include those having a Spatial Light Modulator (SLM), Digital micromirror array or Liquid Crystal on Silicon (LCOS) modulators.

A flow cell or other channel, such a microfluidic channel or microfluidic channels having an input and an output is used to deliver fluids including reagents, such as a polymerase, a nucleotide, cations (or precursors thereof) whether in an active or inactive state and other appropriate reagents and washes to particular locations on a substrate within the flow cell, such as within a reaction chamber including a reaction region or reaction zone. A desired location, such as a grid point on a substrate or array, can be provided with reaction conditions to facilitate covalent binding of a nucleotide to an initiator sequence, an existing nucleotide or an existing oligonucleotide. Certain reaction conditions as described herein can be provided at the reactive site to prevent further attachment of an additional nucleotide at the same location. Then, reaction conditions to facilitate covalent binding of a nucleotide to an existing nucleotide can be provided to the same location in a method of making an oligonucleotide at that desired location. One of skill will recognize that reaction conditions will be based on dimensions of the substrate reaction region, reagents, concentrations, reaction temperature, and the structures used to create and deliver the reagents and washes. According to certain aspects, pH, light, electrons and other reactants and reaction conditions can be optimized for the use of TdT to add a dNTP to an existing nucleotide or oligonucleotide in a template independent manner.

The disclosure provides use of cation modulation methods in the synthesis of nucleic acids using enzymes, such as a template independent polymerase, which enzymatically adds Deoxynucleotide Triphosphates (dNTPs) to the 3′ OH terminus of ssDNA primer in the absence of a template sequence. The environmental context can be modulated such that a desired ssDNA sequence is synthesized. One example of a Template independent DNA polymerase is Terminal deoxynucleotidyl Transferase (TdT). Reversible terminators may be used and are dNTP analogs that are modified to inhibit subsequent enzymatic extension by DNA polymerases, and may contain labels, such as fluorophores, that produce a signal reporting the identity of the incorporated nucleotide. However, methods described herein to modulate presence of active cations at the reaction region need not utilize reversible terminator dNTPs.

Terms and symbols of nucleic acid chemistry, biochemistry, genetics, and molecular biology used herein follow those of standard treatises and texts in the field, e.g., Komberg and Baker, DNA Replication, Second Edition (W.H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Eckstein, editor, Oligonucleotides and Analogs: A Practical Approach (Oxford University Press, New York, 1991); Gait, editor, Oligonucleotide Synthesis: A Practical Approach (IRL Press, Oxford, 1984); and the like.

### Nucleic Acids and Nucleotides

As used herein, the terms “nucleic acid molecule,” “nucleic acid sequence,” “nucleic acid fragment” “oligonucleotide”, “polynucleotide” and “oligomer” are used interchangeably and are intended to include, but are not limited to, a polymeric form of nucleotides that may have various lengths, including either deoxyribonucleotides or ribonucleotides, or analogs thereof.

In general, the terms “nucleic acid molecule,” “nucleic acid sequence,” “nucleic acid fragment,” “oligonucleotide” and “polynucleotide” are used interchangeably and are intended to include, but not limited to, a polymeric form of nucleotides that may have various lengths, either deoxyribonucleotides (DNA) or ribonucleotides (RNA), or analogs thereof. An oligonucleotide is typically composed of a specific sequence of four nucleotide bases: adenine (A); cytosine (C); guanine (G); and thymine (T) (uracil (U) for thymine (T) when the polynucleotide is RNA). According to certain aspects, deoxynucleotide triphosphates (dNTPs, such as dATP, dCTP, dGTP, dTTP) may be used. According to certain aspects, ribonucleotide triphosphates (rNTPs, such as rATP, rCTP, rGTP, rUTP) may be used. According to certain aspects, ribonucleotide diphosphates (rNDPs) may be used.

The term “oligonucleotide sequence” or simply “sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Oligonucleotides may optionally include one or more non-standard nucleotide(s), nucleotide analog(s) and/or modified nucleotides. The present disclosure contemplates any deoxyribonucleotide or ribonucleotide and chemical variants thereof, such as methylated, hydroxymethylated or glycosylated forms of the bases, and the like. According to certain aspects, natural nucleotides are used in the methods of making the nucleic acids. Natural nucleotides lack chain terminating moieties. According to another aspect, the methods of making the nucleic acids described herein do not use terminating nucleic acids or otherwise lack terminating nucleic acids, such as reversible terminators known to those of skill in the art. The methods are performed in the absence of chain terminating nucleic acids or wherein the nucleic acids are other than chain terminating nucleic acids.

Examples of modified nucleotides include, but are not limited to diaminopurine, S2T, 5-fluorouracil, 5-bromouracil, 5-chlorouracil, 5-iodouracil, hypoxanthine, xantine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-D46-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, 2,6-diaminopurine and the like. Nucleic acid molecules may also be modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety or phosphate backbone. Nucleic acid molecules may also contain amine-modified groups, such as aminoallyl-dUTP (aa-dUTP) and aminohexhylacrylamide-dCTP (aha-dCTP) to allow covalent attachment of amine reactive moieties, such as N-hydroxy succinimide esters (NHS).

Modified nucleotide mono, di, tri phosphates and their synthesis methods have been described (Roy, B., Depaix, A., Perigaud, C., & Peyrottes, S, (2016), Recent Trends in Nucleotide Synthesis. Chemical Reviews, 116(14), 7854-7897), which is hereby incorporated by reference in its entirety.

Alternatives to standard DNA base pairs or RNA base pairs in the oligonucleotides of the present disclosure can provide higher density in bits per cubic mm, higher safety (resistant to accidental or purposeful synthesis of natural toxins), easier discrimination in photo-programmed polymerases, or lower secondary structure. Such alternative base pairs compatible with natural and mutant polymerases for de novo and/or amplification synthesis are described in Betz K, Malyshev D A, Lavergne T, Welte W, Diederichs K, Dwyer T J, Ordoukhanian P, Romesberg F E, Marx A (2012) KlenTaq polymerase replicates unnatural base pairs by inducing a Watson-Crick geometry, Nature Chem. Biol. 8:612-614; See Y J, Malyshev D A, Lavergne T, Ordoukhanian P, Romesberg F E. J Am Chem Soc. 2011 Dec. 14; 133(49):19878-88, Site-specific labeling of DNA and RNA using an efficiently replicated and transcribed class of unnatural base pairs; Switzer C Y, Moroney S E, Benner S A. (1993) Biochemistry. 32(39):10489-96. Enzymatic recognition of the base pair between isocytidine and isoguanosine; Yamashige R, Kimoto M, Takezawa Y, Sato A, Mitsui T, Yokoyama S, Hirao I. Nucleic Acids Res. 2012 March; 40(6):2793-806. Highly specific unnatural base pair systems as a third base pair for PCR amplification; and Yang Z, Chen F, Alvarado J B, Benner S A. J Am Chem Soc. 2011 Sep. 28; 133(38):15105-12, Amplification, mutation, and sequencing of a six-letter synthetic genetic system. Other non-standard nucleotides may be used such as described in Malyshev, D. A., et al., Nature, vol. 509, pp. 385-388 (15 May 2014), which are hereby incorporated by reference in their entireties.

### Polymerases

According to one aspect, polymerases are used to build nucleic acid molecules which may represent encoded information which is referred to herein as being recorded in the nucleic acid sequence or the nucleic acid is referred to herein as being storage media. Polymerases are enzymes that produce a nucleic acid sequence, for example, using DNA or RNA as a template. Polymerases that produce RNA polymers are known as RNA polymerases, while polymerases that produce DNA polymers are known as DNA polymerases. Polymerases that incorporate errors are known in the art and are referred to herein as “error-prone polymerases”. Template independent polymerases may be error prone polymerases. Using an error-prone polymerase allows the incorporation of specific bases at precise locations of the DNA molecule. Error-prone polymerases will either accept a non-standard base, such as a reversible chain terminating base, or will incorporate a different nucleotide, such as a natural or unmodified nucleotide that is selectively provided during primer extension.

### Template Independent Polymerases

As used herein, template-independent polymerases, refer to polymerase enzymes which catalyze extension of a target substrate such as a polynucleotide primer strand or growing nucleic acid strand with nucleotides in the absence of a polynucleotide template. Template independent polymerases where the polynucleotide primer is DNA are known as Template independent DNA polymerases. Template independent polymerases where the polynucleotide primer is RNA are known as Template independent RNA polymerases. Template independent polymerases may accept a broad range of nucleotide polyphosphate substrates, which may be single stranded nucleotide polyphosphate substrates. Template independent DNA polymerase are defined to include all enzymes with activity classified by the Enzyme commission number EC 2.7.7.31 (See, enzyme—ExPASy: SIB Bioinformatics Resource Portal, EC 2.7.7.31).

According to certain aspects of the invention the template independent DNA polymerase is a terminal deoxynucleotidyl transferase (TdT) of the polX family of DNA polymerases. TdT may also be referred to as DNA nucleotidylexotransferase, (DNTT) or simply terminal transferase. According to further aspects of the disclosure, TdT is of mammalian origin, for example, from bovine or murine sources. Further description of TdT is provided in Biochim Biophys Acta., May 2010; 1804(5): 1151-1166, hereby incorporated by reference in its entirety. TdT creates polynucleotide strands by catalyzing the addition of nucleotides to the 3′ terminus of a DNA molecule (i.e., a substrate for the enzyme) in the absence of a template. The preferred substrate of TdT is a 3′-overhang, but it can also add nucleotides to blunt or recessed 3′ ends. Cobalt (Co²⁺) is a cofactor, however the enzyme catalyzes reaction upon Mg²⁺, Zn²⁺, and Mn²⁺ administration in vitro. Nucleic acid initiator fragments or sequences may be 4 or 5 nucleotides or longer and may be single stranded or double stranded. Double stranded initiators may have a 3′ overhang or they may be blunt ended or they may have a 3′ recessed end. Preferred nucleotides are dTTP, dATP, dGTP, dCTP. TdT can catalyze incorporation of many modified nucleotides.

According to certain aspects of the disclosure, the template independent DNA polymerase is a terminal deoxynucleotidyl transferase of the archaeo-eukaryotic primase (AEP) superfamily Exemplary terminal transferases are described (Guilliam, T. A., Keen, B. A., Brissett, N. C., & Doherty, A. J, (2015), Primase-polymerases are a functionally diverse superfamily of replication and repair enzymes, Nucleic Acids Research, 43(14), 6651-64), which is hereby incorporated by reference in its entirety.

In further aspects of the disclosure, the terminal transferase is PolpTN2, a DNA primase-polymerase protein encoded by the pTN2 plasmid from Thermococcus nautilus. In further aspects of the contemplated disclosure a C-terminal truncation of PolpTN2 may be used, such as Δ₃₁₁₋₉₂₃ (Sukhvinder Gill et al., A highly divergent archaeo-eukaryotic primase from the Thermococcus nautilus plasmid, pTN2, Nucleic Acids Research, Volume 42, Issue 6, Pp. 3707-3719, http://doi.org/10.1093/nar/gkt1385)

In further aspects of the disclosure, the terminal transferase is PriS, a primase S subunit from the kingdom Archea. For example: DNA primase complex of p41-p46 or PriSL as described in the following:

Pyrococcus furiosus (Lidong Liu et al., The Archaeal DNA Primase BIOCHEMICAL CHARACTERIZATION OF THE p41-p46 COMPLEX FROM Pyrococcus furiosus, The Journal of Biological Chemistry, 276, 45484-45490, 2001, doi:10.1074/jbc.M106391200),

Thermococcus kodakaraensis (Wiebke Chemnitz Galal et al., Characterization of DNA Primase Complex Isolated from the Archaeon, Thermococcus kodakaraensis, The Journal of Biological Chemistry 287, 16209-16219, 2012, doi: 10.1074/jbc.M111.338145),

Sulfolobus solfataricus (Si-houy Lao-Sirieix, et al., The Heterodimeric Primase of the Hyperthermophilic Archaeon Sulfolobus solfataricus Possesses DNA and RNA Primase, Polymerase and 3′-terminal Nucleotidyl Transferase Activities, Journal of Molecular Biology, Volume 344, Issue 5, 2004, Pages 1251-1263, http://dx.doi.org/10.1016/j.jmb.2004.10.018),

Pyrococcus horikoshii (Eriko Matsui et al., Distinct Domain Functions Regulating de Novo DNA Synthesis of Thermostable DNA Primase from Hyperthermophile Pyrococcus horikoshii, Biochemistry, 2003, 42 (50), pp 14968-14976, DOI: 10.1021/bi0355560), and

Archaeoglobus fulgidus (Stanislaw K. Jozwiakowski, et al., Archaeal replicative primases can perform translesion DNA synthesis, PNAS, 2015, vol. 112, no. 7, E633-E638, doi: 10.1073/pnas.1412982112), each of which is hereby incorporated by reference in its entirety.

In further aspects of the disclosure, the terminal transferase is an archeal nonhomologous end joining archaeo-eukaryotic primase.

In further aspects of the disclosure, the terminal transferase is a mammalian Pol 0 as described (Tatiana Kent, et al., Mechanism of microhomology-mediated end-joining promoted by human DNA polymerase θ, Nature Structural & Molecular Biology, Vol. 22, 230-237, (2015), doi:10.1038/nsmb.2961), hereby incorporated by reference in its entirety.

In further aspects of the disclosure, the terminal transferase is a Eukaryotic PrimPol, for example, human primPol have been described (Sara García-Gómez, et al., PrimPol, an Archaic Primase/Polymerase Operating in Human Cells, Molecular Cell, Volume 52, Issue 4, 2013, Pages 541-553, http://dx.doi.org/10.1016/j.molce1.2013.09.025), (Thomas A. Guilliam, et al., Human PrimPol is a highly error-prone polymerase regulated by single-stranded DNA binding proteins, Nucl. Acids Res., (2015), 43 (2): 1056-1068, doi: 10.1093/nar/gku1321), each of which is hereby incorporated by reference in its entirety.

Those skilled in the art will recognize that DNA polymerases utilize cations, such as divalent metal cations, for catalysis of primer extension with deoxyribonucleotide polyphosphates, such as deoxyribonucleotide triphosphates (dNTPs) and the like. Those skilled in the art will further recognize that conditions for template independent DNA synthesis by DNA polymerases require a polynucleotide sequence containing 3′ terminal hydroxyl, one or more cations, such as a plurality of divalent metal cations, a nucleotide polyphosphate, and a DNA polymerase enzyme with template independent DNA polymerase activity.

### Supports and Attachment

In certain exemplary embodiments, one or more oligonucleotide sequences described herein are immobilized on a support (e.g., a solid and/or semi-solid support) which may also be referred to as a “substrate”. In certain aspects, an oligonucleotide sequence can be attached to a support using one or more of the phosphoramidite linkers described herein. Suitable supports include, but are not limited to, slides, beads, chips, particles, strands, gels, sheets, tubing, spheres, containers, capillaries, pads, slices, films, plates and the like. In various embodiments, a solid support may be biological, nonbiological, organic, inorganic, or any combination thereof. Supports of the present invention can be any shape, size, or geometry as desired. For example, the support may be square, rectangular, round, flat, planar, circular, tubular, spherical, and the like. When using a support that is substantially planar, the support may be physically separated into regions, for example, with trenches, grooves, wells, or chemical barriers (e.g., hydrophobic coatings, etc.). Supports may be made from glass (silicon dioxide), metal, ceramic, polymer or other materials known to those of skill in the art. Supports may be a solid, semi-solid, elastomer or gel. In certain exemplary embodiments, a support is a microarray. As used herein, the term “microarray” refers in one embodiment to a type of array that comprises a solid phase support having a substantially planar surface on which there is an array of spatially defined non-overlapping regions or sites that each contain an immobilized hybridization probe or oligonucleotide primer. “Substantially planar” means that features or objects of interest, such as probe sites, on a surface may occupy a volume that extends above or below a surface and whose dimensions are small relative to the dimensions of the surface. For example, beads disposed on the face of a fiber optic bundle create a substantially planar surface of probe sites, or oligonucleotides disposed or synthesized on a porous planar substrate create a substantially planar surface. Spatially defined sites may additionally be “addressable” in that its location and the identity of the immobilized probe or primer at that location are known or determinable.

Oligonucleotides immobilized on microarrays include nucleic acids that are generated in or from an assay reaction. Typically, the oligonucleotides or polynucleotides on microarrays are single stranded and are covalently attached to the solid phase support, usually by a 5′-end or a 3′-end. In certain exemplary embodiments, probes or primers are immobilized via one or more cleavable linkers. The density of non-overlapping regions containing nucleic acids in a microarray is typically greater than 100 per cm², and more typically, greater than 1000 per cm². Microarray technology relating to nucleic acid probes is reviewed in the following exemplary references: Schena, Editor, Microarrays: A Practical Approach (IRL Press, Oxford, 2000); Southern, Current Opin. Chem. Biol., 2: 404-410 (1998); Nature Genetics Supplement, 21:1-60 (1999); and Fodor et al, U.S. Pat. Nos. 5,424,186; 5,445,934; and 5,744,305, hereby incorporated by reference in their entireties.

Methods of immobilizing oligonucleotides to a support are known in the art (beads: Dressman et al. (2003) Proc. Natl. Acad. Sci. USA 100:8817, Brenner et al. (2000) Nat. Biotech. 18:630, Albretsen et al. (1990) Anal. Biochem. 189:40, and Lang et al. Nucleic Acids Res. (1988) 16:10861; nitrocellulose: Ranki et al. (1983) Gene 21:77; cellulose: Goldkorn (1986) Nucleic Acids Res. 14:9171; polystyrene: Ruth et al. (1987) Conference of Therapeutic and Diagnostic Applications of Synthetic Nucleic Acids, Cambridge U.K.; teflon-acrylamide: Duncan et al. (1988) Anal. Biochem. 169:104; polypropylene: Polsky-Cynkin et al. (1985) Clin. Chem. 31:1438; nylon: Van Ness et al. (1991) Nucleic Acids Res. 19:3345; agarose: Polsky-Cynkin et al., Clin. Chem. (1985) 31:1438; and sephacryl: Langdale et al. (1985) Gene 36:201; latex: Wolf et al. (1987) Nucleic Acids Res. 15:2911, hereby incorporated by reference in their entireties). Supports may be coated with attachment chemistry or polymers, such as amino-silane, NHS-esters, click chemistry, polylysine, etc., to bind a nucleic acid to the support.

As used herein, the term “attach” refers to both covalent interactions and noncovalent interactions. A covalent interaction is a chemical linkage between two atoms or radicals formed by the sharing of a pair of electrons (i.e., a single bond), two pairs of electrons (i.e., a double bond) or three pairs of electrons (i.e., a triple bond). Covalent interactions are also known in the art as electron pair interactions or electron pair bonds. Noncovalent interactions include, but are not limited to, van der Waals interactions, hydrogen bonds, weak chemical bonds (i.e., via short-range noncovalent forces), hydrophobic interactions, ionic bonds and the like. A review of noncovalent interactions can be found in Alberts et al., in Molecular Biology of the Cell, 3d edition, Garland Publishing, 1994, hereby incorporated by reference in its entirety.

According to certain aspects, affixing or immobilizing nucleic acid molecules to the substrate is performed using a covalent linker including an attachment moiety such as oxidized 3-methyl uridine, an acrylyl group and hexaethylene glycol. In addition to the attachment of linker sequences to the molecules of a pool, for example, for use in directional attachment to the support, a restriction site or regulatory element (such as a promoter element, cap site or translational termination signal), is, if desired, joined with the members of the pool. Linkers can also be designed with chemically reactive segments which are optionally cleavable with agents such as enzymes, light, heat, pH buffers, and redox reagents. Such linkers can be employed to pre-fabricate an in situ solid-phase inactive reservoir of a different solution-phase primer for each discrete feature. Upon linker cleavage, the primer would be released into solution for PCR, perhaps by using the heat from the thermocycling process as the trigger.

It is also contemplated that affixing of nucleic acid molecules to the support is performed via hybridization of the members of the pool to nucleic acid molecules that are covalently bound to the support.

Immobilization of nucleic acid molecules to the support matrix according to the invention is accomplished by any of several procedures. Direct immobilizing via the use of terminal tags or terminal moieties that can be attached to the support, such as 5′-terminal tags or 3′-terminal tags bearing chemical groups suitable for covalent linkage to the support, hybridization of single-stranded molecules of a pool of nucleic acid molecules to oligonucleotide primers already bound to the support, or the spreading of the nucleic acid molecules on the support accompanied by the introduction of primers, added either before or after plating, that may be covalently linked to the support, may be performed. Where pre-immobilized primers are used, they are designed to capture a broad spectrum of sequence motifs (for example, all possible multimers of a given chain length, e.g., hexamers), nucleic acids with homology to a specific sequence or nucleic acids containing variations on a particular sequence motif. Alternatively, the primers encompass a synthetic molecular feature common to all members of a pool of nucleic acid molecules, such as a linker sequence.

Alternatively, hexaethylene glycol is used to covalently link nucleic acid molecules to nylon or other support matrices (Adams and Kron, 1994, U.S. Pat. No. 5,641,658, hereby incorporated by reference in its entirety). In addition, nucleic acid molecules are crosslinked to nylon via irradiation with ultraviolet light. While the length of time for which a support is irradiated as well as the optimal distance from the ultraviolet source is calibrated with each instrument used due to variations in wavelength and transmission strength, at least one irradiation device designed specifically for crosslinking of nucleic acid molecules to hybridization membranes is commercially available (Stratalinker, Stratagene). It should be noted that in the process of crosslinking via irradiation, limited nicking of nucleic acid strands occurs. The amount of nicking is generally negligible, however, under conditions such as those used in hybridization procedures. In some instances, however, the method of ultraviolet crosslinking of nucleic acid molecules will be unsuitable due to nicking. Attachment of nucleic acid molecules to the support at positions that are neither 5′- nor 3′-terminal also occurs, but it should be noted that the potential for utility of an array so crosslinked is largely uncompromised, as such crosslinking does not inhibit hybridization of oligonucleotide primers to the immobilized molecule where it is bonded to the support, nor does the crosslinking inhibit the incorporation of nucleotides to the growing end of the immobilized primers.

### Reagents and Reagent Delivery Systems

As used herein, the term “reagent” refers to an aqueous solution containing some or all of the following components: nucleotides, enzymes, enzyme substrates, ions, buffers, surfactants, or reducing agents. In certain compositions of reagents, the nucleotide is a dNTP, rNTP, or rNDP, and the nucleotides may be provided in a concentration range of 100 micromolar up to 100 millimolar. In certain embodiments of reagent compositions, the enzyme is a template independent DNA polymerase, and may be provided at concentrations in the range of 1 nanogram per microliter up to 500 nanograms per microliter. In certain embodiments of reagent compositions, monovalent ions and divalent ions can be used. In certain exemplary reagent compositions, the monovalent ion is a cation, and may include the some or all of the alkali metals, such as but not limited to Li⁺, Na⁺, or K⁺ which may be provided in the range of 10 micromolar to 500 millimolar. In certain exemplary reagent compositions, the monovalent ion is an anion and may include some or all of the halide anions, such as but not limited to: F⁻, Cl⁻, Br⁻, and I⁻ which may be provided in the range of 10 micromolar to 500 millimolar. In certain exemplary reagent compositions, the divalent ion is a cation and may include the some or all of the transition metals, such as but not limited to: Co²⁺, Mn²⁺, Zn²⁺ and Mg²⁺ which may be provided in the range of 10 micromolar to 100 millimolar. In certain exemplary reagent compositions, the divalent ion is a cation and may include the some or all of the alkaline earth elements such as Mg²⁺, Ca²⁺, S²⁺ and Ba²⁺, which may be provided in the range of 10 micromolar to 100 millimolar. In certain compositions of reagents, the buffer is any buffer stable in the pH range of 6 to 9, and greater than 0.5 units away from the pKI of the enzyme at room temperature. In certain embodiments of reagent compositions, the buffer may include but is not limited to the following molecules: MES, Bis-Tris, ADA, ACES, PIPES, HEPPSO, MOPSO, BES, MOPS, TES, HEPES, DIPSO, TAPSO, POPSO, Trizma, Tricine, Bicine, TAPS, AMPSO, CHES, and CAPSO. In certain embodiments of reagent compositions, the buffer can be provided at a concentration in the range, of 50 micromolar to 500 millimolar. It is understood that the buffers described herein are known to those of skilled in the art, and can be readily identified by a literature search. In certain embodiments of reagent compositions, surfactants can be used to prevent aggregation or nonspecific binding of enzymes, for example, nonionic surfactants such as, but not limited to: triton-X in the range of 0.01% to 1%, TWEEN-20 or TWEEN-80 in the range of 0.01% to 1%, like can be used. In certain embodiments of reagent compositions, reducing agents may be used to prevent aggregation or crosslinking of proteins by disulfide bond formation. In certain compositions of reagents, reducing agents may include but is not limited to DTT, TCEP, BME, and the like provided at concentrations in the range of 100 micromolar to 50 millimolar. Other reagent compositions useful in the present invention are known to those skilled in the art and can be readily identified by a literature search.

According to certain aspects, reagents and washes are delivered such that the reactants are present at a desired location for a desired period of time to, for example, enzymatically and covalently attach a nucleotide to an initiator polynucleotide or an existing polynucleotide (i.e, substrate for the enzyme) attached at the desired location. A selected extension reagent comprising of a single selected nucleotide suitable for extension is pulsed or flowed or deposited at the reaction site where synthesis reaction takes place and then may be optionally followed by delivery of a wash buffer, of similar composition to the extension reagent that does not include the nucleotide. There are contemplated different distributions for the time for binding a nucleotide precursor (dNTP/rNTP/rNDP) and time spent in making the covalent bond with the growing primer 3′ end.

Suitable delivery systems include fluidics systems, microfluidics systems, syringe systems, ink jet systems, pipette systems and other fluid delivery systems known to those of skill in the art. Various flow cell embodiments or flow channel embodiments or microfluidic channel embodiments are envisioned which can deliver separate reagents or a mixture of reagents or washes using pumps or electrodes or other methods known to those of skill in the art of moving fluids through channels or microfluidic channels through one or more channels to a reaction region or vessel where the surface of the substrate is positioned so that the reagents can contact the desired location where a nucleotide is to be added. According to another embodiment, a microfluidic device is provided with one or more reservoirs which include one or more reagents which are then transferred via microchannels to a reaction zone or reaction region or reaction support where the reagents are mixed and the reaction occurs. Such microfluidic devices and the methods of moving fluid reagents through such microfluidic devices are known to those of skill in the art.

An array-based, flow-cell technique may be used, similar to standard synthesis and sequencing procedures known in the art. Starting polynucleotide primers may be immobilized on a support, for example to flat silicon dioxide, at known locations. Locations for creating oligonucleotides can range in number between 100 and 100 million elements per array. A flow cell or other channel, such a microfluidic channel or microfluidic channels having an input and an output is used to deliver fluids including reagents, and washes to particular locations on a substrate within the flow cell, such as within a reaction chamber. A desired location, such as a grid point on a support or substrate or array, can be provided with reaction conditions to facilitate enzymatic and covalent binding of a nucleotide to an initiator sequence, an existing nucleotide or an existing oligonucleotide as described herein. Certain reaction conditions can be provided at the reactive site to prevent further attachment of an additional nucleotide at the same location. Then, reaction conditions to facilitate covalent binding of a nucleotide to an existing nucleotide can be provided to the same location in a method of making an oligonucleotide at that desired location.

The extension reaction solution may also be displaced by a solution containing a different major nucleotide species. Displacement and a turnover of reactants with new reactions can be facilitated further when solid phase extension occurs enclosed in a flow cell. Flow cell geometries are known to those skilled in the art. In a flow cell, modulation of reaction conditions can be performed by flowing different solutions through the reaction chamber.

### Electron Delivery Systems

The present disclosure provides methods and apparatus for the modulation of DNA synthesis reactions, such as in a plurality of array elements. In certain embodiments DNA synthesis reactions may be modulated by provisioning electric potential to one or more electrode features at or near to the solid support. In certain embodiments there are one or more electrodes present at each element in the array. In certain aspects, the electrode makes contact with the reagents such that under an applied potential, an electrochemical reaction can occur at a specified location in the array. In other aspects the electrode does not make contact with the reagent, but provides an electric field normal to the array under an applied potential. In further aspects of the disclosure, the counterelectrode is located proximal to the electrode. In other embodiments of the disclosure, the counterelectrode is located on the opposing surface of the enclosure, flowcell or the like. Various electrode geometries are contemplated, for use in the present invention. Electrodes are contemplated with sizes in the range of 100 nm to 50 microns on the longest axis, square, or circular geometries are preferred, however other geometries may be useful in the present invention.

In certain embodiments, a reactor array is provided on a surface such as a two dimensional surface. In further aspects, the method of control of electrode potential across the array is by an active matrix circuit, for example a CMOS active matrix circuit known to those of skill in the art. In yet further aspects, an active matrix may have row and column registers that control the state and potential of each element in the array. According to certain aspects the potential between the electrode and counter electrode at any element in the array is either 0 volts (off) or 3, 3.3, 5, or 12 Volts (on). In certain aspects of the disclosure the current provided to the electrode array elements is controlled to be within the range of nano amperes to microamperes per electrode. Integrated circuits, and methods of construction therein useful in the present disclosure are described in US patent numbers U.S. Pat. Nos. 6,258,606, 7,267,751, 6,682,936 and 5,341,012, hereby incorporated by reference in their entireties.

### Photon Delivery Systems

The present disclosure provides methods and apparatus for the modulation of DNA synthesis reactions in a plurality of array elements. In certain embodiments of the disclosure, control of reactor array elements is accomplished by the delivery of photons to select locations of the array. Photons may be delivered to specific regions of a reaction array by multiplexed binary illumination systems known to those skilled in the art. According to certain aspects, light sources may comprise coherent or incoherent collimated light sources such as continuous wave lasers, pulsed lasers, mercury or xenon Arc lamps, and other photon light sources known to those skilled in the art. Reaction regions or reaction zones or reaction substrates are illuminated by the light sources describe herein and others known to those of skill in the art. In certain exemplary illumination systems, such as spatially multiplexed illumination systems, such as spatial light modulator arrays, digital micromirror devices (DMD), Liquid Crystal Displays (LCD), Light Emitting Diode (LED) arrays, optical fiber arrays, and acousto optic modulator (AOM) arrays may be used for generating two dimensional or three dimensional light intensity patterns. In certain embodiments of illumination, such as multiplexed illumination, light scanning methods may be used, wherein a light beam or plurality of light beams are scanned serially across individual elements in a reactor array. According to further aspects, the binary modulation of the beam amplitude may be modulated by mechanical shutters, TTL controllers, or other methods known to those skilled in the art. Light provided from light sources provided herein may be directed to array elements in 2 or 3 dimensional space by light scanning systems such as galvano mirrors, MEMS microscanner arrays, and other systems known to those skilled in the art.

### Multiplex Control Methods of DNA Synthesis

By limiting nucleotides available to the template independent DNA polymerase, the incorporation of specific nucleotides into a polynucleotide or plurality of polynucleotides can be regulated. Thus, template independent polymerases are capable of creating nucleic acid sequences de novo. The combination of a template independent DNA polymerase and a primer sequence serves as a writing mechanism for imparting information into a nucleic acid sequence such as when the nucleic acid sequence is used to encode information. This present disclosure provides methods to control the primer extension activity of template independent DNA polymerases such as terminal deoxynucleotidyl transferase (TdT), and other DNA polymerases described herein. One of skill in the art will recognize that reaction conditions will be based on geometry and composition of the enzyme active site, reagents, concentrations, reaction temperature, and the structures used to create and deliver the reagents and washes.

This present disclosure provides methods for control of enzymatic extension of polynucleotides, at a reaction site or at a plurality of reaction sites. According to certain aspects, reaction conditions can be modulated to turn enzymatic primer extension on or off across an array of polynucleotides in the presence of different nucleotide reagents. In conditions where a single nucleotide polyphosphate species is in excess, active template independent DNA polymerase extension of the primer produces a homopolymer sequence composed of the major nucleobase species in solution. The primer extension production can serve as a primer in subsequence extension reactions.

### Control of DNA Synthesis by Polymerase Conformation

The conformational state of the template independent polymerases can be used to modulate DNA synthesis at various array elements. In further aspects, control of DNA synthesis is accomplished by switching enzymes at a reaction site or zone or region from active conformation to an inactive conformation, or from an inactive conformation to an active conformation between nucleotide delivery steps. In a further aspect, a multiplexed control of DNA synthesis is accomplished by switching enzymes at array elements from active conformations to inactive conformations, or from inactive conformations to active conformations between nucleotide delivery steps. In one embodiment of conformational control, a template independent DNA polymerase is modified to contain a unnatural amino acid containing photoisomerizable side chains proximal to the aspartic acid residues comprising the catalytic active site. This allows photoreversible conformational changes of the polymerase to inhibit, or activate, polynucleotide extension in arrays by spatial light patterns having certain wavelengths of light. In further aspects, the photoisomerizable sidechain is an azobenzene moiety incorporated in the polymerase by methods known to those skilled in the art, and are described in Bose M, Groff D, Xie J, Brustad E, Schultz P G, The incorporation of a photoisomerizable amino acid into proteins in E. coli, J Am Chem Soc., 2006, 128(2):388-9, hereby incorporated by reference in its entirety. It is understood by those skilled in the art that azobenzene is a bistable light controllable isomer. It is further appreciated that in the presence of 350 nm light, azobenzene will transition to the Cis conformation, and in 412 nm light azobenzene will transition to the Trans conformation. Therefore, by placing the azobenzene moiety at the active site, enzymes can be rendered active or inactive in a light controlled manner, at any sufficiently illuminated location in an array. This disclosure provides photon delivery methods for multiplexed illumination of photoisomerizable moieties useful in the present disclosure.

In an alternative embodiment of conformational control, electric fields are used to alter the conformational state of the enzymes. It is contemplated that intensities in the range of 30 to 10 kilovolts/cm, enzymes can be irreversibly denatured, provided that the electric field does not exceed the dielectric strength of the reagent medium. In further aspects of the present disclosure, the electrode and counter electrodes form parallel planes that do not contact the reagent medium. Electrode geometries and spacing satisfying the required electric field intensities will be apparent to those skilled in the art. This disclosure provides electric potential delivery methods for multiplexed conformational control of enzymes useful in the present disclosure.

### Control of DNA Synthesis by Spatial Modulation of Reagents

Embodiments of the present disclosure provide that the presence or absence of the template independent polymerases, nucleotides or primers can be used to modulate single stranded enzymatic DNA synthesis. In one embodiment, enzyme substrates such as ssDNA primers (single stranded nucleic acid substrates or primers), are immobilized to a support which may include array elements, and template independent DNA polymerases are spatially modulated. In one embodiment of spatial modulation of enzymes, electric fields are used to engage or disengage the enzyme with the primer via electrophoresis. In further aspects, electrophoresis is provided by introducing electric fields normal to the plane of an array such that each elements in the array can be actuated independently. This disclosure provides electric potential delivery methods for multiplexed control of enzymes useful in the present disclosure. Controlling activity of DNA extension in the presence of specific nucleotides enables multiplex synthesis of a plurality of polynucleotide sequences.

In a separate embodiment of spatial modulation, immobilized nucleic acid molecules may be produced using a device (e.g., any commercially-available inkjet printer, which may be used in substantially unmodified form) which sprays a focused burst of reagent-containing solution onto a support (see, e.g., Castellino, (1997), Genome Res., 7:943-976, hereby incorporated by reference in its entirety). Such a method is currently in practice at Incyte Pharmaceuticals and Rosetta Biosystems, Inc., the latter of which employs “minimally modified Epson inkjet cartridges” (Epson America, Inc.; Torrance, Calif.). The method of inkjet deposition depends upon the piezoelectric effect, whereby a narrow tube containing a liquid of interest (in this case, oligonucleotide synthesis reagents) is encircled by an adapter. An electric charge sent across the adapter causes the adapter to expand at a different rate than the tube, and forces a small drop of liquid reagents from the tube onto a coated slide or other support.

Embodiments of the present disclosure provide that nucleotides can be deposited onto a discrete region of the support, such that each region forms a feature of the array. The desired nucleic acid sequence can be synthesized drop-by-drop at each position, as is true for other methods known in the art. If the angle of dispersion of reagents is narrow, it is possible to create an array comprising many features.

### Control of DNA Synthesis by pH Modulation

Localized pH changes can reversibly or irreversibly modulate the activity of template independent DNA polymerases. The pH of the environment dictates the time averaged protonation state of side chains critical in intermediate stabilization for proton donation or proton abstraction as is useful in the catalysis of phosphodiester bonds by polymerases. It will be apparent to those skilled in the art that protonation state of the active site of template independent polymerases can be used to modulate single stranded DNA synthesis. For example, Ashley et al., Virology 77, 367-375 (1977), hereby incorporated by reference in its entirety, identifies certain reagents and reaction conditions for dNTP addition, such as initiator size, divalent cation and pH. An exemplary template independent DNA polymerase TdT was reported to be active over a wide pH range with an optimal pH of 6.85. In certain embodiments of the disclosure a localized pH changes can be provided by electrochemical methods using electrodes in an array. In further aspects pH may be modulated on electrodes by electrochemical oxidation of hydroquinones, to give benzoquinone, and free protons, thereby increasing the pH locally. pH may also be modulated spatially on electrodes by electrochemical reduction benzoquinone to give hydroquinone, resulting in a local increase in pH. Alternative redox systems for the generation or consumption protons in solutions may be used and will be apparent to those of skill in the art. This disclosure provides electric potential delivery methods for multiplexed control of enzymes useful in the present invention.

### Information Encoding in Polynucleotide Sequences

Embodiments of the present disclosure contemplate encoding information in polynucleotide molecules. In one embodiment, the polynucleotide is single stranded DNA. In certain aspects, information may be encoded in each base up to 2 bits of shannon information (as is understood in the art), if the provided bases are A, T, G, and C. In further aspects, an exemplary 2 bit assignment for each base is A=11, T=10, G=01, C=00. In other aspects, redundant encoding schemes may be used wherein any of the provided bases encode at most 1 bit of information, for example, A and T may be assigned the value 1 and C and G may be assigned the value 0. In further aspects of redundant encoding, polynucleotides may be produced comprised of sequential homopolymers of any base, such that a homopolymer region of any base identity encodes equivalent information as 1 base of the homopolymer region. In an exemplary homopolymer encoding, the polynucleotide sequence: “AAAATTTCCCGGG” (SEQ ID NO: 1) encodes the equivalent information as polynucleotide sequence “ATCG”. In further aspects, homopolymer encodings of length 2-50 nucleotides in length are contemplated for use in the disclosure.

In other aspects, information encoding methods may incorporate error correcting codes as described in Robert N. Grass, et al., Robust Chemical Preservation of Digital Information on DNA in Silica with Error-Correcting Codes, Angewandte Chemie, 2015, Volume 54, Issue 8, Pages 2552-2555, hereby incorporated by reference in its entirety. In further aspects, methods known in the art for encoding information in DNA are described in George M. Church, et al., Next-Generation Digital Information Storage in DNA, Science, 2012, Vol. 337, Issue 6102, pp. 1628, James Bornholt, et al., A DNA-Based Archival Storage System, ACM SIGOPS Operating Systems Review—SIGOPS Member Plus, Volume 50, Issue 2, June 2016, Pages 637-649, hereby incorporated by reference in their entireties.

### Example I

Modulation of Active Cations Modulates TdT Activity to Add a dNTP

In an exemplary embodiment, an exemplary template independent polymerase, terminal deoxynucleotidyl transferase (TdT), is used to catalyze the synthesis of a plurality of polynucleotide sequences in an array. In an exemplary device, primers are immobilized to the surface of a substrate via the 5′ end of the primer. Methods for immobilization of oligonucleotide primers to a substrate are provided herein. In other aspects the polynucleotide array comprises a region of a planar surface of a flow cell. In further aspects the preferred material of the array surface is quartz glass and the flow cell is preferably a rectangular channel having an inlet and outlet. Suitable flow cell geometries and compositions are known to those skilled in the art.

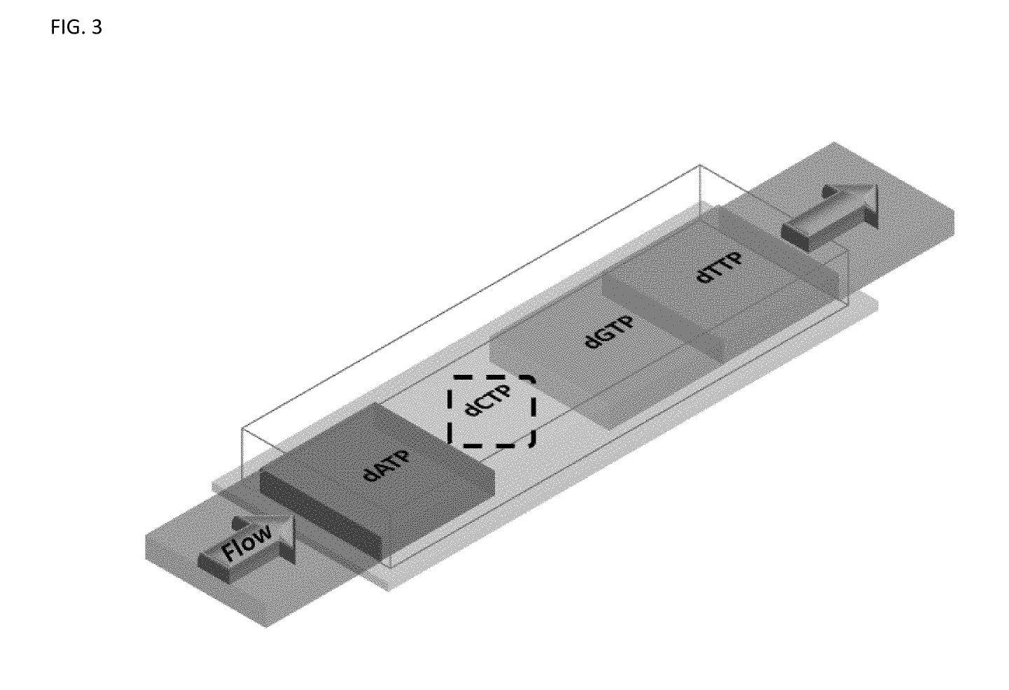

It will be apparent to those of skill in the art that in a flow cell, modulation of reaction conditions can be performed by flowing different solutions through the reaction chamber. It is contemplated that the flowing of extension reagent providing a single nucleotide polyphosphate species in excess will lead to polymerase extension of the primer producing a homopolymer sequence composed of the provided nucleotide. The primer extension product can serve as a primer in subsequence extension reactions. In a preferred embodiment the extension reaction solutions for each base are flowed into the reaction chamber in continuous succession, for example in a cyclic fashion such as, A-->T-->C-->G and so on. FIG. 3 illustrates an exemplary continuous flow synthesis process in an exemplary flow cell geometry. In an alternative embodiment a washing reagent is provided between extension reactions. Therefore, precisely ordered synthesis of homopolymer sequences can be accomplished by performing iterative extension reactions with under a DNA synthesis modulation scheme.

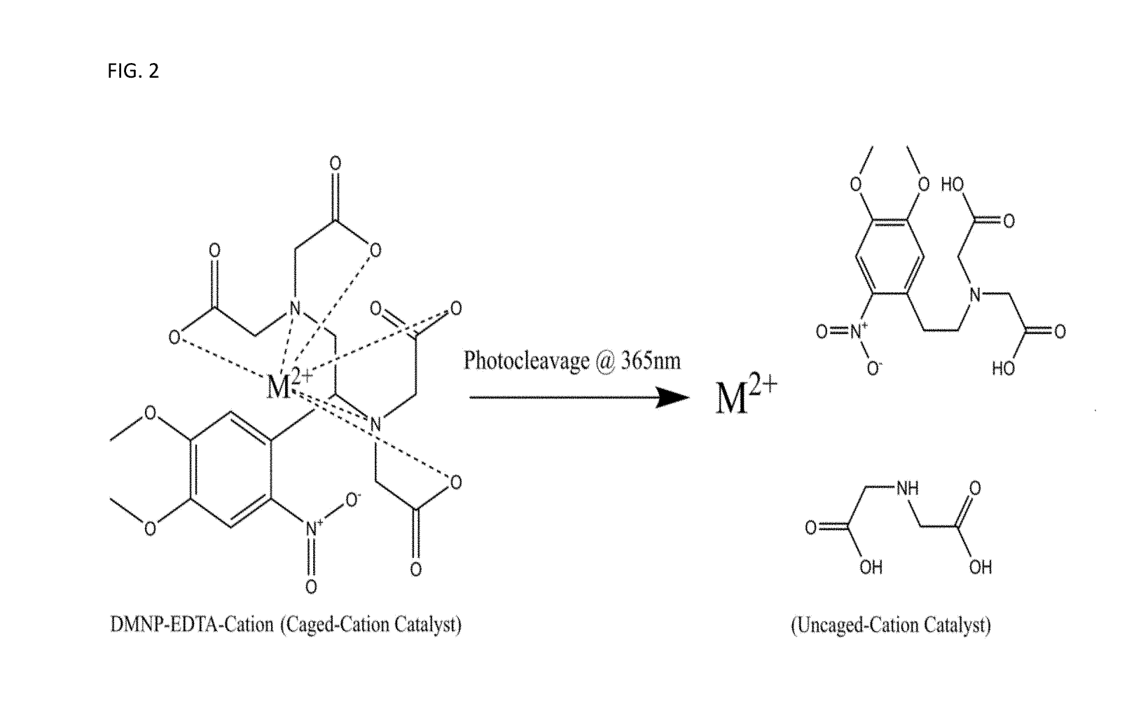

In an exemplary synthesis modulation scheme, the multiplexed DNA synthesis is defined by cycled introduction of nucleotide species in the flow cell, in combination with a spatially multiplexed release of catalytic cofactors of TdT such as Co2+ and Mg2+. In an exemplary embodiment, the catalytic cofactors are provided in the extension reagent as inactive caged-chelation complexes of the catalyst cation species. By actively modulating the free cation concentration in solution, the rate of nucleotide incorporation by TdT can be controlled. In a preferred embodiment, the caged chelator reagent is DMNP-EDTA. It will be apparent to those of skill in the art that UV illumination at 355 nm or 365 nm induces bond cleavage in the ligand backbone DMNP-EDTA, which reduces the denticity of the ligands. FIG. 2 illustrates the UV cleavage reaction of DMNP. DMNP-EDTA's affinity for divalent cations decreases irreversibly by >600,000-fold upon cleavage. Thus, photolysis of DMNP-EDTA complexed with the catalytic cofactor results in a pulse of free divalent cations, which in turn can drive free nucleotide incorporation by TdT in the illuminated regions.

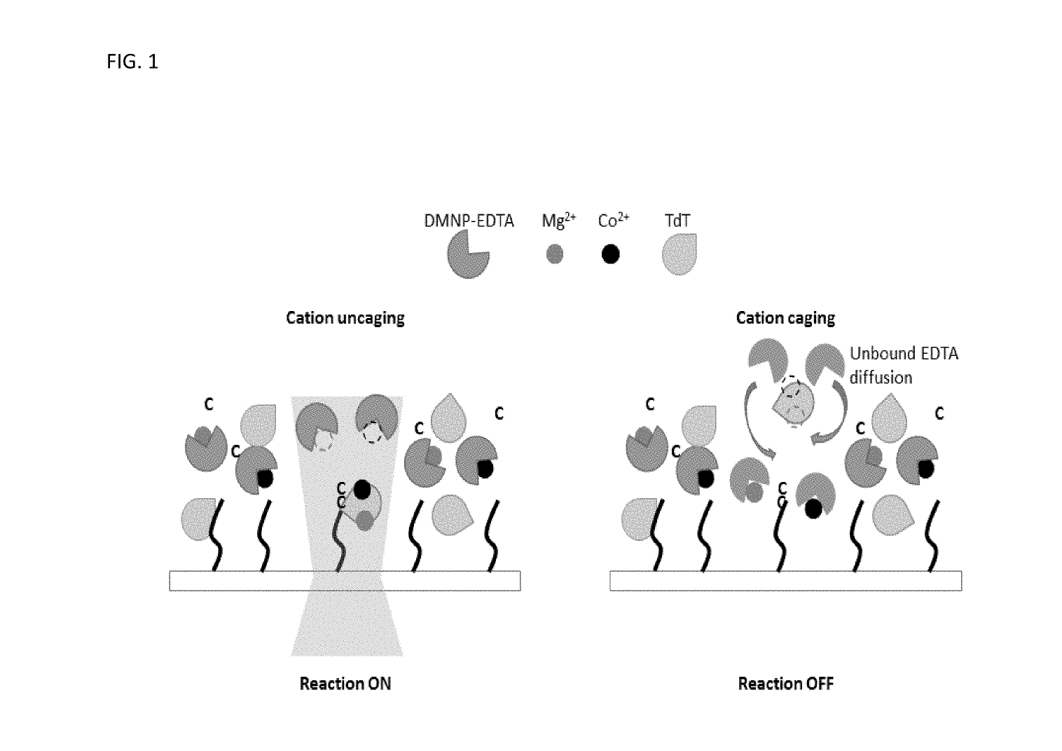

In further aspects of the preferred embodiment, DMNP-EDTA is provided in molar excess of the Mg2+ or Co2+ ion in the extension reagent. In yet further aspects, it is preferable that the caged chelator:catalytic ion ratio fall within the range of 2:1 to 10:1 to the catalytic ion of highest concentration. It is contemplated that, with the caged chelator in excess, the region of illumination will be rapidly quenched by cation recapture post-illumination, due to passive diffusion of free DMNP-EDTA. FIG. 1 illustrates the exemplary process of cation release and capture during and after illumination. As shown in FIG. 1, inactive cations are located at a reaction region. The cations are inactive because they are bound by a photolabile binding or chelating agent. When the reaction region is illuminated by light of a certain wavelength such as UV light corresponding to the photolabile binding or chelating agent, the photolabile binding or chelating reagent releases the cations making them active and available for participating in the enzymatic nucleotide addition reaction. The enzyme and the cation cofactor bind thereby activating the enzyme. The enzyme then releases the cation cofactor into the reaction medium. Photolabile binding or chelating reagent present in the reaction medium at the reaction region or zone binds to the cation cofactor making it unavailable for further participation in the enzymatic nucleotide addition reaction. According to one aspect, the photolabile binding or chelating agent is present in molar excess in the reaction medium at the reaction region or reaction zone.

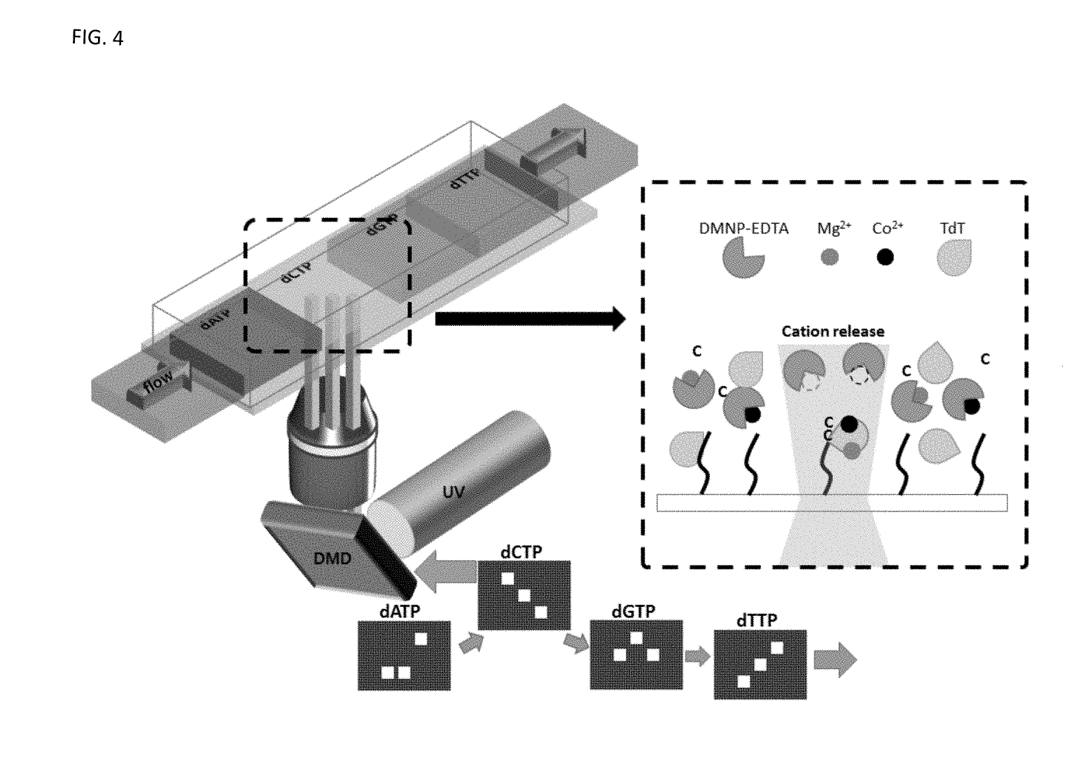

In one embodiment, the UV light is spatially modulated over the array by a digital micromirror device (DMD). In further aspects, the preferred light source is a 355 nm solid state pulsed laser source of 1 W continuous power or higher. FIG. 4 illustrates an exemplary apparatus wherein light provided by a laser is directed from a DMD through an objective onto the synthesis array of a flow cell. By synchronizing the timing of provided uncaging light patterns with each nucleotide extension reagent flow, precisely ordered homopolymer DNA sequences can be made. FIG. 4 illustrates an exemplary series of light patterns and cycled delivery of extension reagents. Taken together, caged cations comprise an exemplary multiplexed synthesis scheme modulated by light useful in various applications.

### Example II

### UV Uncaging of Caged Cations for Enzymatic Synthesis

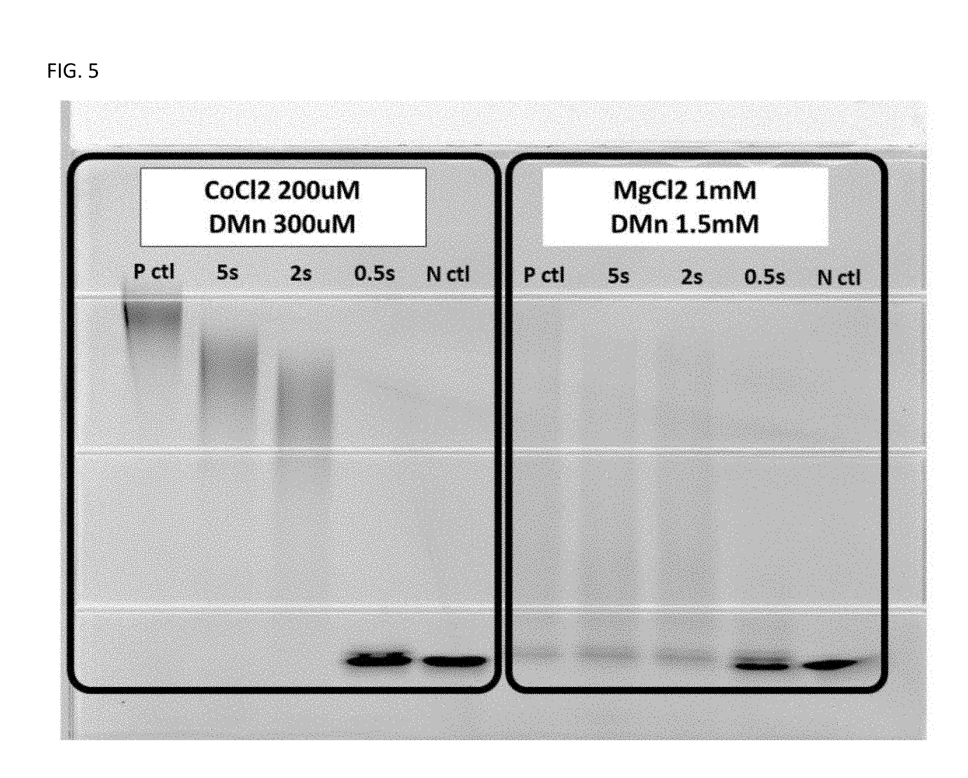

According to one aspect, modulating the illumination time of a caged cation affects the amount of cation that is uncaged and available for enzymatic synthesis. Two experiments were conducted using Co⁺² cations and Mg⁺² cations. In a first experiment, a mixture of CoCl₂ and DMNP-EDTA (“DMn”) was prepared at a ratio of 1 to 1.5 (CoCl₂, 200 μM; DMNP-EDTA, 300 μM). Samples of the mixture were illuminated at a wavelength of 365 nm and at a 500 mW/cm² and at various times of 0.5 seconds, 2 seconds and 5 seconds to determine the effect of illumination time and thus illumination dose on enzymatic synthesis using TdT. A positive control was provided where no DMNP-EDTA chelator was added to the CoCl₂ so as to make all of the Co⁺² cations available for enzymatic synthesis. A negative control was provided where the sample was not subject to illumination. After illumination to uncage or release cation from the chelator, TdT elongation master mix including TdT and dNTP was added to the sample (to avoid the TdT master mix from being illuminated) and the sample was incubated at 37° C. for 30 minutes. The sample was illuminated before adding the TdT master mix so as to remove any effect of illumination on the TdT or other TdT master mix ingredients. FIG. 5 shows a gel image for the first experiment showing TdT activity and enzymatic synthesis of a polynucleotide. The positive control indicates the amount of Co⁺² being fully available for enzymatic synthesis using the TdT master mix. Illumination of the sample of caged Co⁺² cation for 5 seconds produced greater enzymatic synthesis than illumination for 2 seconds which produced greater enzymatic synthesis than illumination for 0.5 seconds and no illumination. The second experiment was carried out using MgCl₂, 1 mM and DMNP-EDTA, 1.5 mM. Illumination of the sample of caged Mg⁺² for 5 seconds produced similar enzymatic synthesis to illumination for 2 seconds, which produced greater enzymatic synthesis than illumination for 0.5 seconds and no illumination. The gel image is presented in FIG. 5 which showed that UV illumination of caged cation cofactors is sufficient to control enzymatic synthesis of polynucleotides, and that cation release is UV dose dependent.

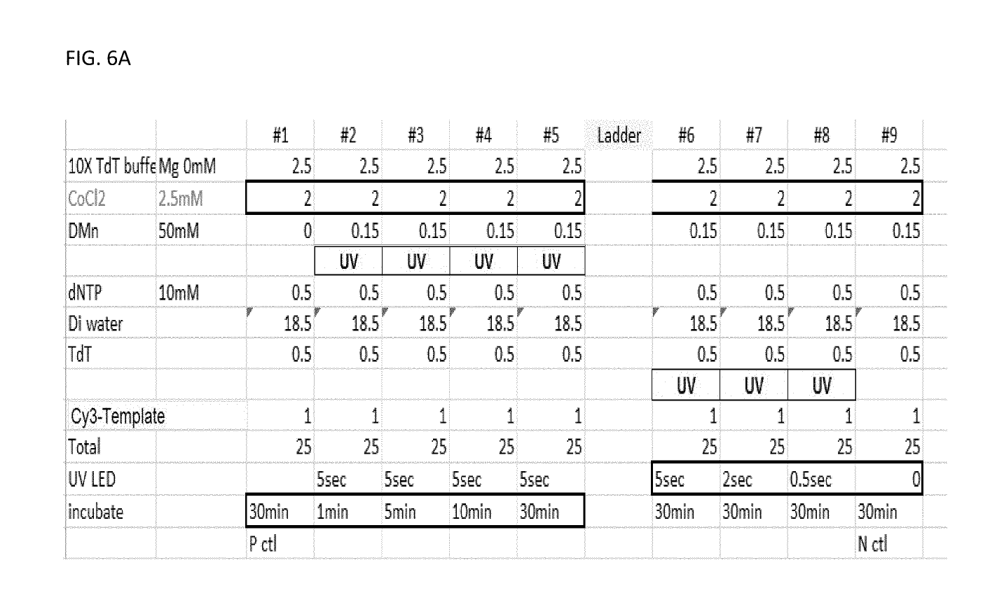

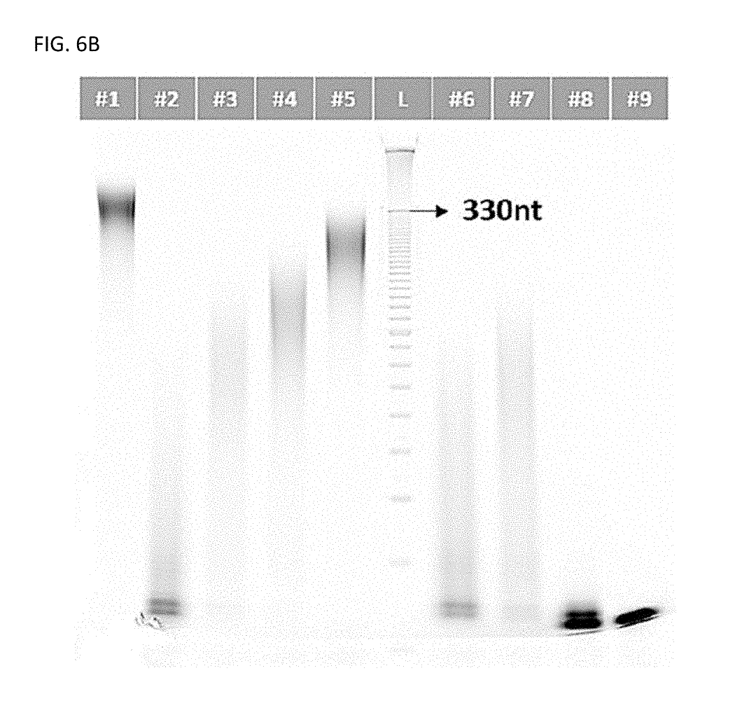

The effect of incubation time for samples illuminated for 5 seconds to uncage or release Co⁺² was determined. The conditions are presented at FIG. 6A lanes 1-5 where samples of a mixture of CoCl₂ and DMNP-EDTA chelator mixture were illuminated for 5 seconds followed by addition of TdT master mix and incubation for 1 minute, 5 minutes, 10 minutes and 30 minutes with a positive control incubated at 30 minutes. As shown in the gel image of FIG. 6B, lanes 1-5 indicate TdT enzymatic activity as a function of incubation time. Lane 2 with a 1 minute incubation time shows relatively short and broad enzymatic product distribution while longer incubation times of 5 minutes, 10 minutes and 30 minutes show more extended products up to 300 nucleotides.

The effect of illumination on a sample including the TdT master mix illuminated for 5 seconds, 2 seconds, 0.5 seconds and a control of 0 seconds was determined using Co⁺² as the cation cofactor. The conditions are presented at FIG. 6A lanes 6-9 indicating illumination after adding the TdT master mix to the sample. Lanes 6-8 show poor elongation products which may be due in part to the UV illumination detrimentally affecting the ability of TdT to engage in enzymatic synthesis. Enzymatic synthesis mediated by cation uncaging is strongly dependent on ideal conditions of illumination dose, i.e., illumination intensity and/or illumination duration.

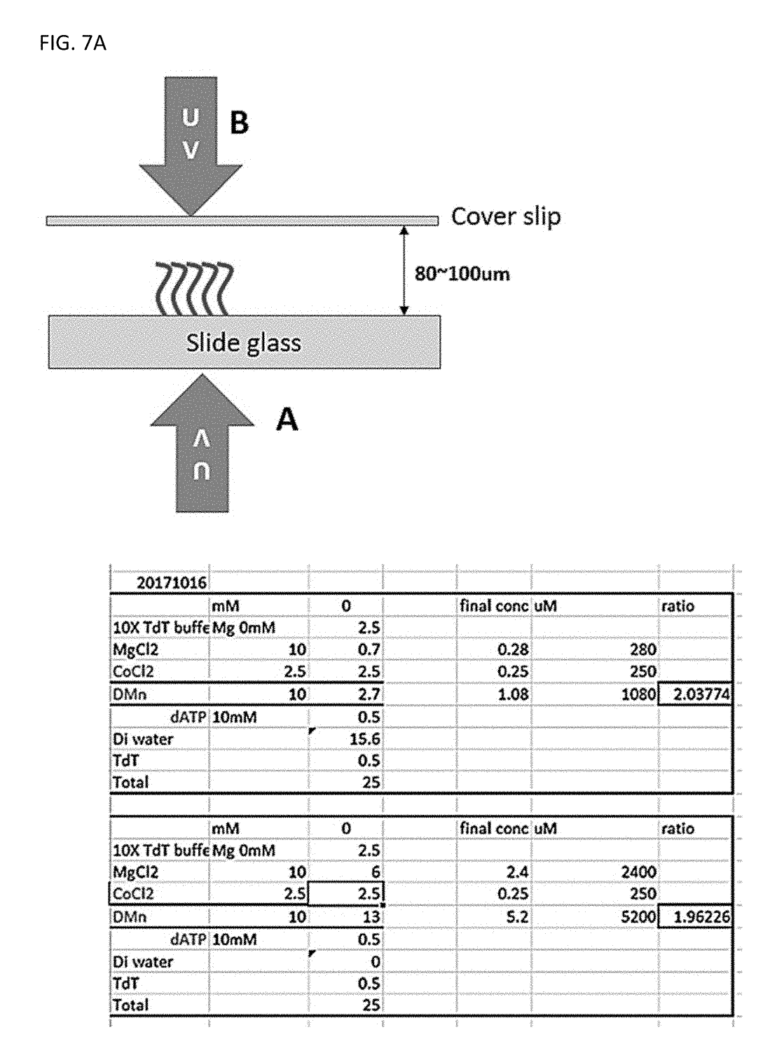

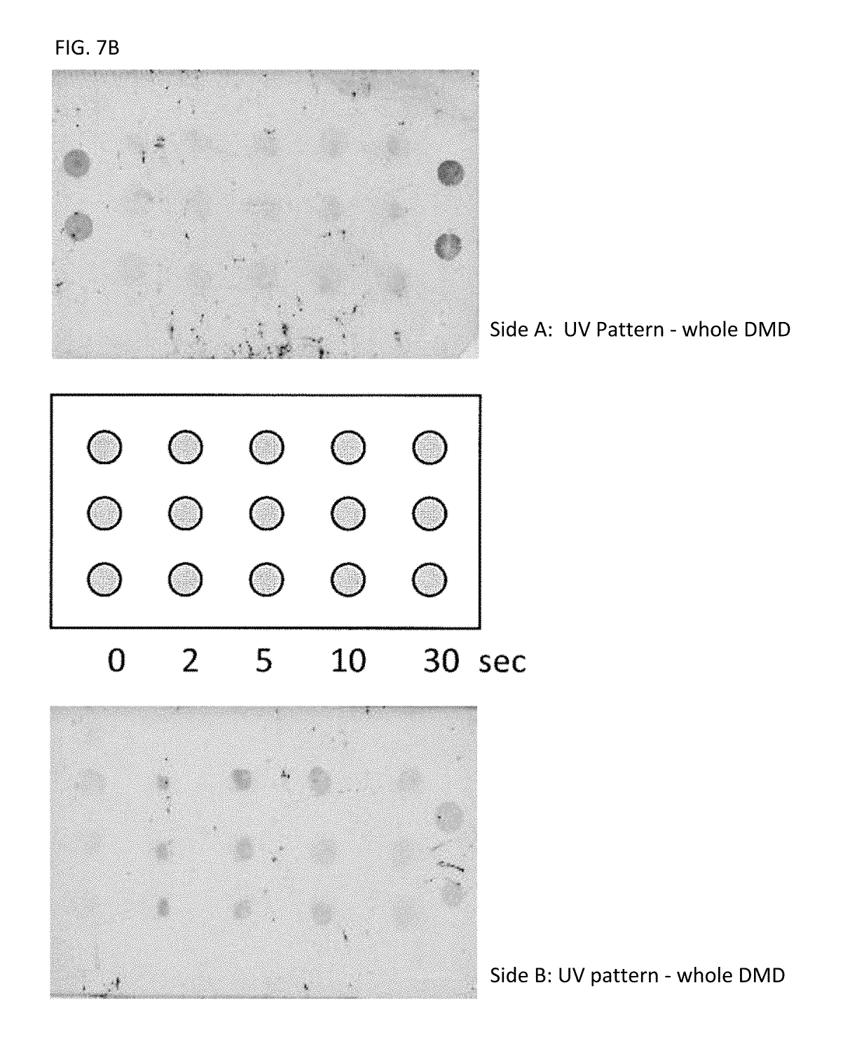

The effect of patterned illumination on the ability to create TdT activation was determined. Patterned UV illumination uncaged cation in a spatially selective matter to activate TdT according to the UV illumination pattern. FIG. 7A depicts a general experimental setup and reaction components and conditions. 5′ end biotinylated initial primer sequences for TdT elongation were functionalized to the surface of a streptavidin coated glass slide. A glass cover slip was positioned between about 80 to 100 μM away from the glass slide. Square pattern UV light reflected from a digital micromirror device was illuminated on the specific area of the glass slide that included chelated cations (DMNP-EDTA-Cobalt ion complex) in order to release cations for use with TdT master mix. Cation was selectively uncaged or released. Illumination time varied from 0 seconds, 2 seconds, 5 seconds, 10 seconds and 30 seconds. The TdT master mix was then provided to the area of the glass slide that was illuminated. FIG. 7B shows a square fluorescence pattern resulting from selective activation of TdT enzyme on the surface of glass slide.

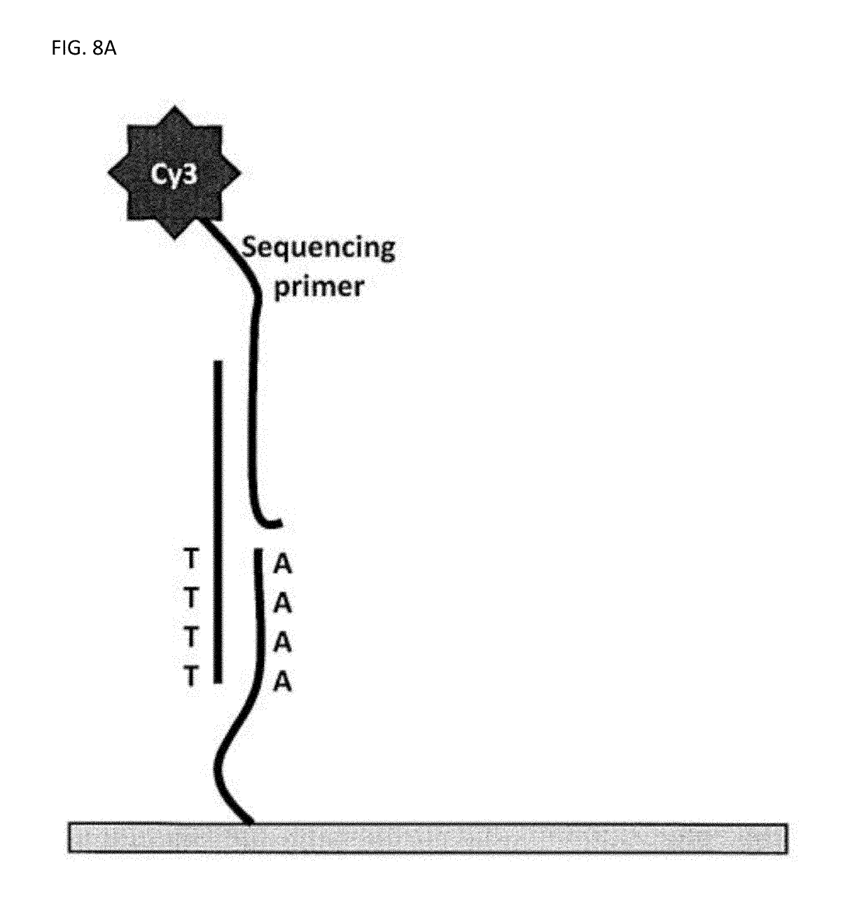

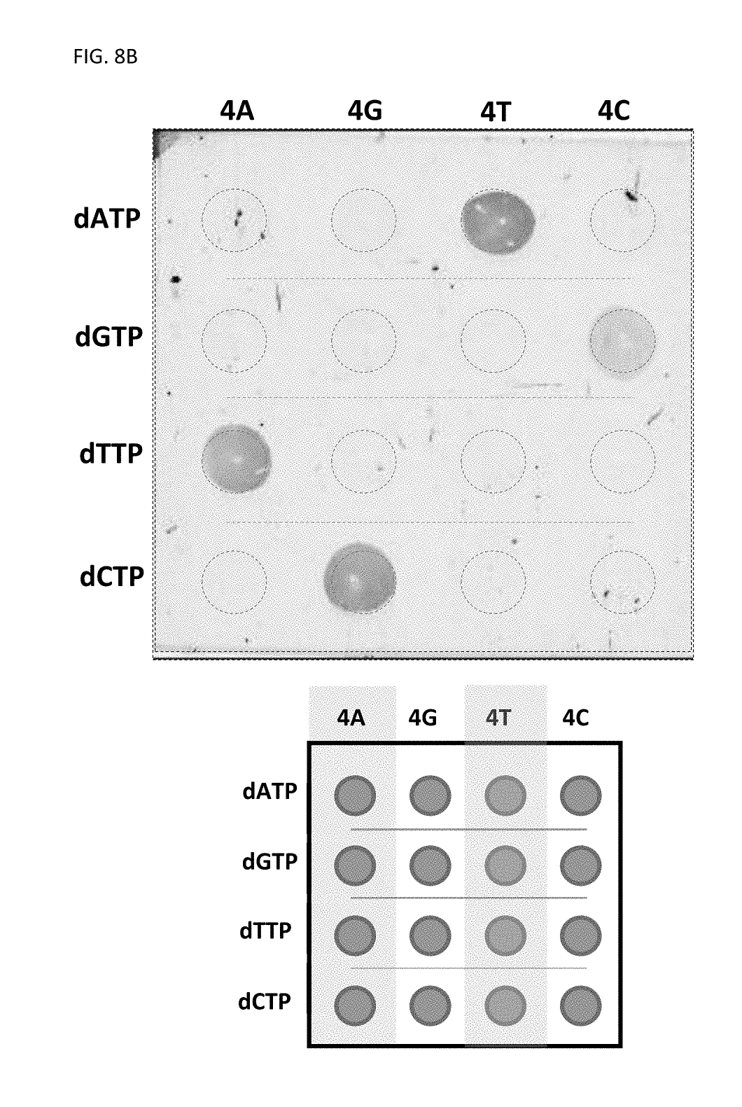

The images of FIG. 7B were created in part using a splint ligation method. According to this aspect, the splint is an oligonucleotide sequence that has a portion complementary to a portion, such as an end portion, of a single stranded oligonucleotide, such as a single stranded DNA (“ssDNA”). The splint hybridizes to the ssDNA to facilitate ligation of a sequencing primer, for example. These aspects of a splint ligation method are shown in schematic in FIG. 8A

According to one aspect, the splint ligation protocol permits labeling immobilized ssDNA oligos with fluorophore probes specific to the terminal nucleotide on immobilized DNA, and allows use of the ligated adapter sequence to generate NGS amplicon Libraries. The ligation probe oligonucleotide sequence facilitates annealing of a reverse PCR primer. According to one aspect, the ligation probe oligo is 5′ phosphorylated and 3′ Dye labeled. Splint oligonucleotides facilitate fast ligation of Dye labeled probe oligos to specific homopolymer sequences of 1 or more nucleotides of a ssDNA bound to a substrate. The splint oligos give modularity to the ligation scheme, so that regions can be labeled independent of the probe oligo sequence and fluorophore.

The 5′ end of the splint oligonucleotide sequence is reverse complementary to the probe sequence. The 3′ end of the splint oligonucleotide sequence includes 1 fixed base (A, T, C or G) at the ligation site for specificity, followed by degenerate N's to support stability of the duplex. In this list of oligos, there are versions with 3 or 4 degenerate “N” at the 3′ end. The oligos are identified by the number of N bases, followed by the fixed base used in the oligo. For example an oligo with 3 N bases preceded by T is called, “N3T”. A universal N4 splint oligo is provided for full surface amplicon preparation. However, instead of using a degenerate sequence, homopolymer sequences of nucleotides, such as 4 nucleotides, may be used to enhance selectivity.

According to a first method, the following components are used: 25 uM Probe and Splint oligo sets in EB buffer (TE buffer), Quick Ligase Kit (NEB), and T4 DNA Ligase Kit High Concentration (NEB). The following reaction mixture is provided on ice: 1 μl Probe Oligo (25 uM) (four probe oligonucleotides containing the following labels; Cy3, Cy5, FAM, Texas Red may be used); 1 μl Splint Oligo (25 uM); 2 μl 10× T4 DNA Ligase Buffer (NEB); 15 μl ddH2O; 1 μl T4 DNA Ligase (High Concentration) (NEB) for a total of 20 μl. The reaction mixture is applied to a flow cell and incubated for 30 minutes or 1 hour at 16 degrees celsius. The flow cell is rinsed thoroughly with Wash Buffer, such as TE, PBS or other wash buffer containing 0.1% Triton-X100.

According to a second method, the following reaction mixture is provided on ice: 1 μl Probe Oligo (25 uM) (four probe oligonucleotides containing the following labels; Cy3, Cy5, FAM, Texas Red may be used); 1 μl Splint Oligo (25 uM); 10 μl 10× Quick Ligase Buffer (NEB); 7 μl ddH2O; 1 μl Quick Ligase (NEB) for a total of 20 μl. The reaction mixture is applied to a flow cell and incubated for 30 minutes or 1 hour at 16 degrees celsius. The flow cell is rinsed thoroughly with Wash Buffer, such as TE, PBS or other wash buffer containing 0.1% Triton-X100.

FIG. 8B shows the results of experiments where splint ligation was used to ligate a labeled probed to a ssDNA having a 4 nucleotide or greater homopolymer end sequence using a splint oligonucleotide sequence having a 4 nucleotide homopolymer complementary sequence.

### Example III

### Embodiments

The present disclosure provides a method of modulating enzymatic synthesis of a polynucleotide including combining reaction reagents including a template-independent polymerase, a selected nucleotide triphosphate, one or more divalent cations bound to a divalent cation chelating agent and excess chelating agent for binding to free divalent cation in an aqueous reaction medium including a target substrate comprising an initiator sequence and having a 3′ terminal nucleotide attached to a single stranded portion, releasing the one or more divalent cations from the chelating agent under conditions wherein one or more of the selected nucleotide triphosphate is covalently added to the 3′ terminal nucleotide such that the selected nucleotide triphosphate becomes a 3′ terminal nucleotide. According to one aspect, the one or more divalent cations is released from the chelating agent in response to light, heat, pH, electrons or chemical reaction. According to one aspect, the method further includes binding free divalent cations with a chelating agent so as to terminate enzymatic synthesis. According to one aspect, the method further includes repeatedly introducing a subsequent selected nucleotide triphosphate to the aqueous reaction medium and releasing the one or more divalent cations from the chelating agent under conditions which enzymatically add one or more of the subsequent selected nucleotide triphosphate to the target substrate until the polynucleotide is formed, and binding free divalent cations with a chelating agent so as to terminate enzymatic synthesis. According to one aspect, the target substrate is immobilized to a reaction site on a support. According to one aspect, the target substrate is immobilized to a reaction site on a support, wherein the reaction site is located within a flow cell channel, a microfluidics channel or a reaction chamber. According to one aspect, the target subtrate is immobilized to a reaction site on a solid or semi-solid support. According to one aspect, a plurality of target substrates are provided at which enzymatic polynucleotide synthesis is carried out. According to one aspect, a plurality of target substrates are provided at a plurality of reaction sites on a solid support at which enzymatic polynucleotide synthesis is carried out. According to one aspect, the reaction reagents are removed from the reaction site by a volume of wash fluid. According to one aspect, one or more of the reaction reagents are delivered by a fluid delivery system comprising fluidics, microfluidics, syringe, ink jet, or pipette systems. According to one aspect, the selected nucleotide triphosphate is a natural nucleotide or a nucleotide analog. According to one aspect, the template-independent polymerase is an error prone template-independent polymerase. According to one aspect, the template-independent polymerase is a template-independent DNA or RNA polymerase. According to one aspect, the template-independent polymerase is a template-independent DNA polymerase. According to one aspect, the template-independent polymerase is a terminal deoxynucleotidyl transferase (TdT). According to one aspect, the template-independent polymerase is a TdT of the polX family of DNA polymerases. According to one aspect, the TdT a mammalian TdT. According to one aspect, the TdT is a member of the archaeo-eukaryotic primase (AEP) superfamily According to one aspect, the TdT is a PolpTN2 or a C-terminal truncated PolpTN2, a PriS, a nonhomologous end joining archaeo-eukaryotic primase, a mammalian Pole), or a eukaryotic PrimPol. According to one aspect, the target substrate is a DNA or an RNA oligonucleotide primer. According to one aspect, the selected nucleotide triphosphate is a deoxyribonucleotide or a ribonucleotide. According to one aspect, the selected nucleotide is a natural nucleotide or a modified nucleotide. According to one aspect, the one or more divalent cations include Co2+, Mn2+, Zn2+ or Mg2+. According to one aspect, the excess chelating agent is present in the reaction medium in a molar excess compared to the free divalent cation in the reaction medium. According to one aspect, the excess chelating agent is present in molar excess of Mg2+ or Co2+ ion in the reaction medium. According to one aspect, chelating agent is within a ratio of greater than 1:1 to 10:1, 3:2 to 10:1, or 2:1 to 10:1 of free divalent cation in highest concentration in the reaction medium.

The disclosure provides a system for enzymatic synthesis of a polynucleotide including a template-independent polymerase, a selected nucleotide triphosphate, and one or more divalent cations bound to a divalent cation chelating agent, and excess chelating agent for binding to free divalent cation in an aqueous medium. According to one aspect, the system further includes a target substrate comprising an initiator sequence and having a 3′ terminal nucleotide attached to a single stranded portion.

The disclosure provides an apparatus for modulating polynucleotide synthesis including a plurality of reaction regions having target substrates immobilized thereto, wherein the target substrates comprise an initiator sequence and having a 3′ terminal nucleotide attached to a single stranded portion, a fluid delivery system in fluid communication with the plurality of reaction regions and configured to provide a controlled flow of one or more reaction reagents to the plurality of reaction regions, wherein the one or more reaction reagents comprise a template independent polymerase, a selected nucleotide triphosphate, one or more divalent cations bound to a divalent cation chelating agent and excess chelating agent for binding to free divalent cation, and a light source for providing light to the plurality of reaction regions.

1. A method of modulating enzymatic synthesis of a polynucleotide comprising
combining reaction reagents including a template-independent polymerase, a selected nucleotide triphosphate, one or more divalent cations bound to a divalent cation chelating agent and excess chelating agent for binding to free divalent cation in an aqueous reaction medium including a target substrate comprising an initiator sequence and having a 3′ terminal nucleotide attached to a single stranded portion,
releasing the one or more divalent cations from the chelating agent under conditions wherein one or more of the selected nucleotide triphosphate is covalently added to the 3′ terminal nucleotide such that the selected nucleotide triphosphate becomes a 3′ terminal nucleotide.

2. The method of claim 1 wherein the one or more divalent cations is released from the chelating agent in response to light, heat, pH, electrons or chemical reaction.

3. The method of claim 1 further comprising binding free divalent cations with a chelating agent so as to terminate enzymatic synthesis.

4. The method of claim 1 further including
repeatedly introducing a subsequent selected nucleotide triphosphate to the aqueous reaction medium and releasing the one or more divalent cations from the chelating agent under conditions which enzymatically add one or more of the subsequent selected nucleotide triphosphate to the target substrate until the polynucleotide is formed, and
binding free divalent cations with a chelating agent so as to terminate enzymatic synthesis.

5. The method of claim 1 wherein the target substrate is immobilized to a reaction site on a support.

6. The method of claim 1 wherein the target substrate is immobilized to a reaction site on a support, wherein the reaction site is located within a flow cell channel, a microfluidics channel or a reaction chamber.

7. The method of claim 1 wherein the target substrate is immobilized to a reaction site on a solid or semi-solid support.

8. The method of claim 1 wherein a plurality of target substrates are provided at which enzymatic polynucleotide synthesis is carried out.

9. The method of claim 1 wherein a plurality of target substrates are provided at a plurality of reaction sites on a solid support at which enzymatic polynucleotide synthesis is carried out.

10. The method of claim 1 wherein the reaction reagents are removed from the reaction site by a volume of wash fluid.

11. The method of claim 1 wherein one or more of the reaction reagents are delivered by a fluid delivery system comprising fluidics, microfluidics, syringe, ink jet, or pipette systems.

12. The method of claim 1 wherein the selected nucleotide triphosphate is a natural nucleotide or a nucleotide analog.

13. The method of claim 1 wherein the template-independent polymerase is an error prone template-independent polymerase.

14. The method of claim 1 wherein the template-independent polymerase is a template-independent DNA or RNA polymerase.

15. The method of claim 1 wherein the template-independent polymerase is a template-independent DNA polymerase.

16. The method of claim 1 wherein the template-independent polymerase is a terminal deoxynucleotidyl transferase (TdT).

17. The method of claim 1 wherein the template-independent polymerase is a TdT of the polX family of DNA polymerases.

18. The method of claim 16 wherein the TdT a mammalian TdT.

19. The method of claim 16 wherein the TdT is a member of the archaeo-eukaryotic primase (AEP) superfamily.

20. The method of claim 16 wherein the TdT is a PolpTN2 or a C-terminal truncated PolpTN2, a PriS, a nonhomologous end joining archaeo-eukaryotic primase, a mammalian Poiθ, or a eukaryotic PrimPol.

21. The method of claim 1 wherein the target substrate is a DNA or an RNA oligonucleotide primer.

22. The method of claim 1 wherein the selected nucleotide triphosphate is a deoxyribonucleotide or a ribonucleotide.

23. The method of claim 1 wherein the selected nucleotide is a natural nucleotide or a modified nucleotide.

24. The method of claim 1 wherein the one or more divalent cations include Co2+, Mn2+, Zn2+ or Mg2+.

25. The method of claim 1 wherein the excess chelating agent is present in the reaction medium in a molar excess compared to the free divalent cation in the reaction medium.

26. The method of claim 1 wherein the excess chelating agent is present in molar excess of Mg2+ or Co2+ ion in the reaction medium.

27. The method of claim 1 wherein chelating agent is within a ratio of greater than 1:1 to 10:1, 3:2 to 10:1 or 2:1 to 10:1 of free divalent cation in highest concentration in the reaction medium.

28. A system for enzymatic synthesis of a polynucleotide comprising
a template-independent polymerase,
a selected nucleotide triphosphate, and
one or more divalent cations bound to a divalent cation chelating agent, and
excess chelating agent for binding to free divalent cation in an aqueous medium.

29. The system of claim 28 further including a target substrate comprising an initiator sequence and having a 3′ terminal nucleotide attached to a single stranded portion.

30. An apparatus for modulating polynucleotide synthesis comprising
a plurality of reaction regions having target substrates immobilized thereto, wherein the target substrates comprise an initiator sequence and having a 3′ terminal nucleotide attached to a single stranded portion,
a fluid delivery system in fluid communication with the plurality of reaction regions and configured to provide a controlled flow of one or more reaction reagents to the plurality of reaction regions, wherein the one or more reaction reagents comprise a template independent polymerase, a selected nucleotide triphosphate, one or more divalent cations bound to a divalent cation chelating agent and excess chelating agent for binding to free divalent cation, and
a light source for providing light to the plurality of reaction regions.
